(19) United States
(12) Patent Application Publication
(10) Pub. No.: US 2017/0189484 A1
(43) Pub. Date: Jul. 6, 2017

(54) CEACAM5 PEPTIDES FOR CROHN'S DISEASE

(71) Applicant: ICAHN SCHOOL OF MEDICINE AT MOUNT SINAI, New York, NY (US)

(72) Inventors: Giulia RODA, New York, NY (US); Lloyd MAYER, New York, NY (US)

(21) Appl. No.: 15/308,595

(22) PCT Filed: May 1, 2015
(86) PCT No.: PCT/US2015/028864

§ 371 Date: Nov. 2, 2016

Related U.S. Application Data
(60) Provisional application No. 61/988,091, filed on May 2, 2014.

(51) Int. Cl. A61K 38/17; C12N 5/0783; G01N 33/50; A61K 35/17; A61K 9/00

(52) U.S. Cl. CPC A61K 38/1764; A61K 35/17; A61K 9/0019; G01N 33/505; C12N 5/0637; C12N 2501/599; G01N 2333/70596; G01N 2333/70517; G01N 2800/065

(57) ABSTRACT

The present invention relates to small peptides, derived from the N-terminal domain of CEACAM5 (carcinoembryonic antigen family member 5), with the ability to stimulate the suppressive activity of CD8+ T cells in Crohn's disease. Pharmaceutical formulations, methods to treat patients with Crohn's disease, and methods to identify candidate peptides for treatment of Crohn's disease patients, are also disclosed.

This application claims the benefit of U.S. Provisional Patent Application Ser. No. 61/988,091, filed May 2, 2014, which is hereby incorporated by reference in its entirety.

### FIELD OF THE INVENTION

The present invention relates to small peptides, derived from the N-terminal domain of CEACAM5 (carcinoembryonic antigen family member 5), with the ability to stimulate the suppressive activity of CD8+ T cells in Crohn's disease.

### BACKGROUND OF THE INVENTION

Crohn's disease is a chronic relapsing transmural inflammatory condition that most commonly affects the intestinal wall, but may also occur in any part of the gastrointestinal tract. Crohn's disease occurs only when there is a genetic predisposition and/or an abnormal function of the intestinal immune system. Patients with Crohn's disease suffer from an inappropriate response of the innate and/or adaptive immune system to the intestinal microbiota. In addition to weight loss, patients mainly suffer chronic diarrhea and recurrent right iliac fossa abdominal pain.

In healthy individuals, interactions between intestinal epithelial cells (IECs) and lamina propria lymphocytes give rise to a population of CD8+ T cells with suppressor functions (Ts). Ts cells are known to be critically important T cell subsets for homeostasis of the immune system and are primarily responsible for dampening immune responses. Impairment of suppressor T cell activities can lead to mucosal inflammation. IECs therefore play an important role in the maintenance of the intestinal homeostasis (Dahan et al., Immuno. Rev. 215:243-53 (2007)).

Initial studies demonstrated that IECs derived from Inflammatory Bowel Disease (IBD) patients fail to activate CD8+ regulatory T (TrE) cells from Crohn's disease patients due to a defect in CEACAM5 expression. As a result, lamina propria CD8+ T suppressor cells from Crohn's disease patients have reduced suppressor activity (Allez et al., Ann N Y Acad Sci. 1029:22-35 (2004); Roda et al., Inflamm Bowel Dis. 15(12):1775-83 (2009); Rabinowitz et al., Gastroenterology 144(3):601-12 (2013)).

More recent studies show that, during homeostasis, human CD8+ T cells are activated and expanded when cultured with isolated human IECs through a complex of the nonclassic class I molecule CD1d with CEACAM5 on IECs and CD8α and the T cell receptor (TCR) on the surface of CD8+ T cells (Roda et al., Mucosal Immunol. 7(3): 615-24 (2014)). CEACAM5 is the only CEA family member expressed on IECs to interact with CD1d (id.). This unique set of interactions facilitates antigen presentation by CD to T cells and allows the subsequent activation of CD8+ regulatory T cells that possess potent suppressive function (id.).

Evidence of an immunomodulatory role of CEACAM5 in acquisition of suppressive properties by CD8+ T cells upon their activation by CEACAM5 peptide has also been demonstrated. Furthermore, the interaction between CEACAM5 and CD8α, and the interaction between CEACAM5 and CD1d, were both further characterized, showing that CEACAM5 interacts with CD1d through the B3-domain and with CD8α through the N-domain. Moreover, it was shown that the region within residues 70 and 81 in the CECAM5 N-domain, where the first highly conserved glycosylation site is located, is crucial for CEACAM5/CD8α binding and CD8α activation (id.).

An explanation for the lack of the suppressive CD8+ T cell phenotype in Crohn's disease, however, was absent. It was also unknown whether CEACAM5, or peptides derived therefrom, could be used to improve the suppressive activity of CD8+ T cells in Crohn's disease.

The present invention is directed to overcoming these and other deficiencies in the art.

### SUMMARY OF THE INVENTION

A first aspect of the present invention relates to a method of stimulating suppressive activity in CD8+ T cells. This method involves providing a population of CD8+ T cells having reduced suppressive activity and contacting the provided population of CD8+ T cells with a peptide from a CEACAM5 N-terminal domain under conditions effective to stimulate suppressive activity in the provided population of CD8+ T cells.

A second aspect of the present invention relates to a method of treating Crohn's disease in a subject. This method involves selecting a subject with Crohn's disease and administering a peptide from a CEACAM5 N-terminal domain to a population of CD8+ T cells of the subject, where the population of CD8+ T cells has reduced suppressive activity, under conditions effective to treat Crohn's disease in the subject.

A third aspect of the present invention relates to a method of identifying candidate peptides potentially suitable for treating Crohn's disease in a subject. This method involves providing a collection of candidate peptides from a CEACAM5 N-terminal domain and a population of CD8+ T cells having reduced suppressive activity. A peptide from the collection of peptides is contacted with the population of CD8+ T cells under conditions effective to permit interaction between them. Peptides which interact with the population, as a result of said contacting, are identified as candidate peptides potentially suitable for treating Crohn's disease in a subject.

A fourth aspect of the present invention relates to a pharmaceutical formulation comprising a peptide from a CEACAM5 N-terminal domain and a pharmaceutically acceptable carrier. The N-terminal domain consists essentially or consists of: (i) residues 45-70 of the amino acid sequence of SEQ ID NO: 1, (ii) residues of 62-70 of the amino acid sequence of SEQ ID NO: 1, or (iii) residues of 45-81 of the amino acid sequence of SEQ ID NO: 1.

Provided herein is the use of small CEACAM5 peptide fragments in the stimulation of CD8+ T cells with suppressive activity. These peptides can be used to treat patients with Inflammatory Bowel Disease, especially Crohn's disease patients, who lack suppressive CD8+ T cells as a consequence of a defect in CEACAM5 expression in their intestinal epithelial cells. Among other benefits, it is expected that the smaller peptides would be more useful in treating Crohn's disease than the full-length CEACAM5 peptide (or the full N-terminal domain). Smaller peptides, for example, are more suitable for oral delivery than larger peptides, due to the greater ability of smaller peptides to cross the intestinal barrier. Smaller peptides also often have fewer side effects due to their more targeted activity.

Other features and advantages of the invention will be apparent from the following detailed description and claims.

### BRIEF DESCRIPTION OF DRAWINGS

FIGS. 1A-B are histograms relating to the effect of CEACAM5 on the suppressive activity of lamina propria CD8+ T cells isolated from colon cancer (FIG. 1A) or Crohn's disease (FIG. 1B) biopsies.

FIG. 2 shows an overlapping peptide library of the CEACAM5 N-domain. The CEACAM5 N-domain (residues 1-107 (SEQ ID NO: 1)) was dissected into 20 individual peptides (peptides 1-20 (respectively, SEQ ID NOs: 2-21)) with an offset number of 5.

FIGS. 3A-E relate to the evaluation of the ability of the full CEACAM5 library of peptides (i.e., peptides 1-20) or Pools 1-3 to activate CD8+ T cells. Quantification of p-LcK western blots is presented using ImageJ software. FIG. 3A is a graph of the relative fluorescence (normalized to actin expression) detected by western blot after incubation of peripheral blood CD8+ T cells in either the presence or absence (“unstimulated”) of purified CEACAM5, OKT8, or the full library of peptides after 0, 5, 10, or 15 minutes. FIG. 3B is a graph of the percent detection of phosphorylated LcK (“p-LcK”) (normalized to OKT8 incubation) at 5 minutes following incubation of CD8+ T cells with purified CEACAM5, OKT8, or the full library of peptides. FIGS. 3C-E are graphs of the percent detection of p-LcK (normalized to OKT8 incubation) at 5 minutes (FIG. 3C), 10 minutes (FIG. 3D), or 15 minutes (FIG. 3E) following incubation of CD8+ T cells with purified CEACAM5, OKT8, Pool 1 (peptides 1-6), Pool 2 (peptides 7-11), or Pool 3 (peptides 12-20). FIG. 3A is a representative graph from a single experiment. Data in FIGS. 3B-E are representative of five independent experiments. (**p-value <0.01)

FIGS. 4A-G relate to the evaluation of the ability of individual CEACAM5 library peptides (i.e., peptides 1-20) to activate CD8+ T cells. FIGS. 4A, 4C, and 4E are graphs of the relative fluorescence (normalized to actin expression) detected by western blot after incubation of peripheral blood CD8+ T cells in either the presence or absence (“unstimulated”) of purified CEACAM5, OKT8, the full library of peptides, or each individual peptide after the indicated time periods. FIGS. 4B, 4D, and 4F-G are graphs of the percent detection of phosphorylated LcK (“p-LcK”) (normalized to OKT8 incubation) at the indicated time points following incubation. FIGS. 4A-B show the evaluation of individual peptides in Pool 1 (peptides 1-6). FIGS. 4C-D show the evaluation of individual peptides in Pool 2 (peptides 7-11). FIGS. 4E-G show the evaluation of individual peptides in Pool 3 (peptides 12-20). FIGS. 4A, 4C, and 4E are representative graphs from a single experiment. Data in FIGS. 4B, 4D, and 4F-G are representative of five independent experiments. (**p-value <0.01; ***p-value <0.001)

FIGS. 5A-F relate to the ability of the full CEACAM5 library of peptides (i.e., peptides 1-20) or Pools 1-3 to stimulate the suppressor activity of CD8+ T cells. FIGS. 5A-E are histograms of the proliferation of CFSE-labeled CD4+ T cells in the presence of unstimulated CD8+ T cells (light grey) overlain over histograms of their proliferation in the presence of CD8+ T cells stimulated by the full-length CEACAM5 peptide (FIG. 5A), the full N-peptide library (FIG. 5B), Pool 1 (FIG. 5C), Pool 2 (FIG. 5D), or Pool 3 (FIG. 5E) (x-axis: CFSE intensity; y-axis: # cells). FIG. 5F is a graph of the percent suppression of CD4+ T cell proliferation normalized against the unstimulated CD8+ T cells. FIGS. 5A-E are each representative histograms from a single experiment. Data in FIG. 5F are representative of four independent experiments.

FIGS. 6A-H relate to the ability of peptides 10-15 to stimulate the suppressor activity of CD8+ T cells. FIGS. 6A-G are histograms of the proliferation of CFSE-labeled CD4+ T cells in the presence of unstimulated CD8+ T cells (light grey) overlain over histograms of their proliferation in the presence of CD8+ T cells stimulated by the full-length CEACAM5 peptide (FIG. 6A), peptide 10 (FIG. 6B), peptide 11 (FIG. 6C), peptide 12 (FIG. 6D), peptide 13 (FIG. 6E), peptide 14 (FIG. 6F), or peptide 15 (FIG. 6G) (x-axis: CFSE intensity; y-axis: # cells). FIG. 6H is a graph of the percent suppression of CD4+ T cell proliferation normalized against the unstimulated CD8+ T cells. FIGS. 6A-G are each representative histograms from a single experiment. Data in FIG. 6H are representative of four independent experiments.

FIGS. 7A-G relate to the ability of peptides a-d to stimulate the suppressor activity of CD8+ T cells. FIG. 7A is an alignment showing peptides a-d (SEQ ID NOs: 22, 23, 24, and 25, respectively) relative to residues 37-71 of the CEACAM5 N-terminal domain. FIGS. 7B-F are histograms of the proliferation of CFSE-labeled CD4+ T cells in the presence of unstimulated CD8+ T cells (light grey) overlain over histograms of their proliferation in the presence of CD8+ T cells stimulated by the full-length CEACAM5 peptide (FIG. 7B), peptide a (FIG. 7C), peptide b (FIG. 7D), peptide c (FIG. 7E), or peptide d (FIG. 7F) (x-axis: CFSE intensity; y-axis: # cells). FIG. 7G is a graph of the percent suppression of CD4+ T cell proliferation normalized against the unstimulated CD8+ T cells. FIGS. 7B-F are each representative histograms from a single experiment. Data in FIG. 7G are representative of three independent experiments.

FIGS. 8A-B relate to the ability of peptide 12 to induce phosphorylation of CD8-associated LcK. CD8+ T cells stimulated with OKT8 or with CEACAM5 at the indicated time points were evaluated by flow cytometry for p-LcK staining. Data are representative of four independent experiments.

FIGS. 9A-C relate to suppression assays using CEACAM5 and peptide 12. FIGS. 9A-B are graphs of the proliferation of CD4+ T cells in presence of unactivated Crohn's disease lamina propria CD8+ T cells (grey) or Crohn's disease lamina propria CD8+ T cells activated with CEACAM5 (FIG. 9A, black) or peptide 12 (FIG. 9B, black). FIG. 9C is a graph showing the average suppression percentage observed in suppression assays with normal (“NL”) and Crohn's disease (“CD”) lamina propria CD8+ T cells (“LP CD8”) with CEACAM5 (sum of 5 experiments) or peptide 12 (“P12”) (sum of 3 experiments). Suppression percentages were calculated in respect to the proliferation of CD4+ T cells in the presence of normal, unstimulated CD8+ T cells.

FIGS. 10A-B are graphs of cytokine IL10 production in normal lamina propria CD8+ T cells (left three bars) or CD4+ T cells (control, right three bars) that were unstimulated or stimulated with OKT8 or either CEACAM5 (FIG. 10A) or peptide 12 (FIG. 10B).

### DETAILED DESCRIPTION OF THE INVENTION

The present invention relates to a method of stimulating suppressive activity in CD8+ T cells. This method involves providing a population of CD8+ T cells having reduced suppressive activity and contacting the provided population of CD8+ T cells with a peptide from a CEACAM5 N-terminal domain under conditions effective to stimulate suppressive activity in the provided population of CD8+ T cells.

CD8+ T cells are a subset of immune effector cells generated in the thymus and characterized by the expression of the T cell receptor (TCR) in addition to CD8, a dimeric co-receptor surface molecule composed of a single CD8α chain and a single CD8β chain. In all aspects of the present invention, the CD8+ T cells are preferably regulatory T cells, more preferably suppressor T cells.

Suitable CD8+ T cells according to all aspects of the present invention include, without limitation, peripheral blood CD8+ T cells and lamina propria CD8+ T cells. In at least one embodiment, the CD8+ T cells are CD8+ T cells from a patient with Crohn's disease.

In all aspects of the present invention involving a population CD8+ T cells, the population can consist of a single type of CD8+ T cell or a mixture of different types.

As will be apparent to those skilled in the art, CD8+ T cells having reduced suppressive activity as used herein include inactivated CD8+ T cells as well as activated CD8+ T cells that nevertheless have an impaired ability to suppress the proliferation of CD4+ T cells.

The suppressive activity of CD8+ T cells can be evaluated using any suitable method known in the art. Such methods include, without limitation, measuring the level of phosphorylation of CD8α-associated LcK kinase, evaluating the effect of the cells on CD4+ T cell proliferation, and/or measuring IL10 production. These methods can be used to compare the suppressive activity of a CD8+ T cell population to that of a positive control (for example, CD8+ suppressor T cells from healthy individuals, or CD8+ T cells that have been activated, for example, with OKT8, full-length CEACAM5, etc.), to identify a population of CD8+ T cell that have reduced suppressive activity. In at least one embodiment, the CD8+ T cells having reduced suppressive activity are CD8+ T cells that have a percent reduction of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, relative to a positive control.

As will be apparent to those skilled in the art, stimulation of suppressor activity as used herein includes any increase in the suppressive activity of the CD8+ T cells. In at least one embodiment, the CD8+ T cells exhibit an increase in suppressive activity of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

In all aspects of the present invention, populations of CD8+ T cells can be provided using any suitable method known in the art. For example, techniques for isolating CD8+ T cells from a human sample and expanding them in culture are well-known in the art. Alternative techniques for producing CD8+ T cells in vitro, for example by initiating the differentiation of precursor cells in culture, are also well-known. (See, e.g., Allez et al., Gastroenterology 123:1516-26 (2002), which is hereby incorporated by reference in its entirety).

CEACAM family members are typically cell membrane associated glycoproteins and are part of the immunoglobulin superfamily. CEACAM5 (GenBank Accession No. NP_001278413, which is hereby incorporated by reference in its entirety) is expressed on IECs and attaches to the cell membrane via a GPI-anchor. CEACAM5 is a marker of IEC differentiation and plays a role in cell adhesion, signal transduction, and innate immunity. In all aspects of the present invention, the CEACAM5 N-terminal domain can have, for example, the amino acid sequence set forth in SEQ ID NO: 1 (see FIG. 2). In at least one embodiment, the CEACAM5 N-terminal domain consists essentially or consists of: (i) residues 45-70 of the amino acid sequence of SEQ ID NO: 1, (ii) residues 70-81 of the amino acid sequence of SEQ ID NO: 1, (iii) residues 62-70 of the amino acid sequence of SEQ ID NO: 1, or (iv) residues 45-81 of the amino acid sequence of SEQ ID NO: 1.

Suitable peptides from a CEACAM5 N-terminal domain according to this and all aspects of the present invention include, without limitation, (i) peptides consisting essentially or consisting of four to about 26 contiguous amino acids of residues 45-70 of the amino acid sequence of SEQ ID NO: 1, (ii) peptides consisting essentially or consisting of four to about 12 contiguous amino acids of residues 70-81 of the amino acid sequence of SEQ ID NO: 1, (iii) peptides consisting essentially or consisting of four to about 9 contiguous amino acids of residues 62-70 of the amino acid sequence of SEQ ID NO: 1, and (iv) peptides consisting essentially or consisting of four to about 37 contiguous amino acids of residues 45-81 of the amino acid sequence of SEQ ID NO: 1. In at least one embodiment, the peptide is selected from the group consisting of peptides 1-20 and peptides a-d. In at least one embodiment, the peptide is selected from the group consisting of peptide 10, peptide 11, peptide 12, peptide 13, peptide 14, peptide 15, peptide b, peptide c, and peptide d. In at least one embodiment, the peptide comprises the sequence SGREIIYPN (SEQ ID NO: 26).

Preferably, the peptide has a minimum length of 4, 5, 6, 7, 8, 9, 10, 11, 12, 13, 14, or 15 amino acids and a maximum length of 4, 5, 6, 7, 8, 9, 10, 11, 12, 13, 14, or 15 amino acids. Combinations of each of these minimum and maximum lengths are expressly contemplated. In a preferred embodiment, the peptide has 4-15 amino acids. In a preferred embodiment the peptide has 4-10 amino acids. In a preferred embodiment the peptide has 4-8 amino acids.

In all aspects of the present invention directed to methods involving contacting a population of CD8+ T cells with a peptide, contacting can be carried out using methods that will be apparent to the skilled artisan, and can be done in vitro or in vivo.

One approach for delivering agents into cells involves the use of liposomes. Basically, this involves providing a liposome which includes agent(s) to be delivered, and then contacting the target cell, tissue, or organ with the liposomes under conditions effective for delivery of the agent into the cell, tissue, or organ.

This liposome delivery system can also be made to accumulate at a target organ, tissue, or cell via active targeting (e.g., by incorporating an antibody or hormone on the surface of the liposomal vehicle). This can be achieved according to known methods.

An alternative approach for delivery of protein- or polypeptide-containing agents involves the conjugation of the desired agent to a polymer that is stabilized to avoid enzymatic degradation of the conjugated protein or polypeptide. Conjugated proteins or polypeptides of this type are described in U.S. Pat. No. 5,681,811 to Ekwuribe, which is hereby incorporated by reference in its entirety.

Yet another approach for delivery of agents involves preparation of chimeric agents according to U.S. Pat. No. 5,817,789 to Heartlein et al., which is hereby incorporated by reference in its entirety. The chimeric agent can include a ligand domain and the agent (e.g., a peptide of the invention). The ligand domain is specific for receptors located on a target cell. Thus, when the chimeric agent is delivered intravenously or otherwise introduced into blood or lymph, the chimeric agent will adsorb to the targeted cell, and the targeted cell will internalize the chimeric agent.

Peptides for use in the present invention may be delivered directly to the targeted cell/tissue/organ. Additionally and/or alternatively, the peptides may be administered to a non-targeted area along with one or more agents that facilitate migration of the peptides to (and/or uptake by) a targeted tissue, organ, or cell. As will be apparent to one of ordinary skill in the art, the peptide itself can be modified to facilitate its transport to a target tissue, organ, or cell, including its transport across the intestinal barrier; and/or to facilitate its uptake by a target cell (e.g., its transport across cell membranes).

In vivo administration can be accomplished either via systemic administration to the subject or via targeted administration to affected tissues, organs, and/or cells, as described infra. Typically, the therapeutic agent (i.e., a peptide of the present invention) will be administered to a patient in a vehicle that delivers the therapeutic agent(s) to the target cell, tissue, or organ. Typically, the therapeutic agent will be administered as a pharmaceutical formulation, such as those described infra.

Exemplary routes of administration include, without limitation, orally, topically, transdermally, parenterally, subcutaneously, intravenously, intramuscularly, intraperitoneally, intraventricularly, and intralesionally; by intratracheal inoculation, aspiration, airway instillation, aerosolization, nebulization, intranasal instillation, oral or nasogastric instillation, intraperitoneal injection, intravascular injection, intravenous injection, intra-arterial injection (such as via the pulmonary artery), intramuscular injection, and intrapleural instillation; by application to mucous membranes (such as that of the nose, throat, bronchial tubes, genitals, and/or anus); and by implantation of a sustained release vehicle.

For use as aerosols, a peptide of the present invention in solution or suspension may be packaged in a pressurized aerosol container together with suitable propellants, for example, hydrocarbon propellants like propane, butane, or isobutane with conventional adjuvants. The peptides for use in the present invention also may be administered in a non-pressurized form.

Exemplary delivery devices include, without limitation, nebulizers, atomizers, liposomes (including both active and passive drug delivery techniques) (Wang & Huang, Proc. Nat'l Acad. Sci. USA 84:7851-55 (1987); Bangham et al., J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; U.S. Pat. No. 5,059,421 to Loughrey et al.; Wolff et al., Biochim. Biophys. Acta 802:259-73 (1984), each of which is hereby incorporated by reference in its entirety), transdermal patches, implants, implantable or injectable protein depot compositions, and syringes. Other delivery systems which are known to those of skill in the art can also be employed to achieve the desired delivery of the peptide to the desired organ, tissue, or cells in vivo to effect this aspect of the present invention.

Contacting (including in vivo administration) can be carried out as frequently as required and for a duration that is suitable to provide the desired effect. For example, contacting can be carried out once or multiple times, and in vivo administration can be carried out with a single sustained-release dosage formulation or with multiple (e.g., daily) doses.

The amount to be administered will, of course, vary depending upon the particular conditions and treatment regimen. The amount/dose required to obtain the desired effect may vary depending on the agent, formulation, cell type, culture conditions (for ex vivo embodiments), the duration for which treatment is desired, and, for in vivo embodiments, the individual to whom the agent is administered.

Effective amounts can be determined empirically by those of skill in the art. For example, this may involve assays in which varying amounts of the peptide of the invention are administered to cells in culture and the concentration effective for obtaining the desired result is calculated. Determination of effective amounts for in vivo administration may also involve in vitro assays in which varying doses of agent are administered to cells in culture and the concentration of agent effective for achieving the desired result is determined in order to calculate the concentration required in vivo. Effective amounts may also be based on in vivo animal studies.

Another aspect of the present invention relates to a method of treating Crohn's disease in a subject. This method involves selecting a subject with Crohn's disease and administering a peptide from a CEACAM5 N-terminal domain to a population of CD8+ T cells of the subject, where the population of CD8+ T cells has reduced suppressive activity, under conditions effective to treat Crohn's disease in the subject.

In this and all aspects of the present invention that relate to Crohns' disease, the disease can be a mild form or an acute form. In some preferred embodiments, the disease is a mild form of Crohn's disease. In other preferred embodiments, the disease is an acute form of Crohn's disease. In all cases, the disease is mediated, at least partially, by a reduction in CD8+ T cell suppressive activity.

Treating according to this aspect of the present invention includes, for example, increasing the number of activated CD8+ T cells in the subject, improving the suppression of CD4+ T cell proliferation in the subject, decreasing inflammation of the intestinal mucosa in the subject, reducing the subject's blood plasma levels of C-reactive protein (a marker of inflammation), and/or alleviating one or more symptoms associated with the disease, such as decreasing weight loss/increasing weight gain, reducing the frequency and/or severity of abdominal pain, and/or reducing the frequency and/or severity of chronic diarrhea. As will be appreciated by the skilled artisan, treating includes both complete recovery as well as a partial alleviation of one or more symptoms of the disease.

Preferably, the selected subject is a human. In at least one embodiment, the selected subject has intestinal epithelial cells that are defective in CEACAM5 expression.

Suitable CEACAM5 N-terminal domains, suitable peptides, and suitable populations of CD8+ T cells having reduced suppressive activity, include those noted above.

Administering according to this aspect of the present invention may be carried out in vivo or ex vivo.

In the case of in vivo administration, the peptides can be administered orally, parenterally, for example, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes, such as, that of the nose, throat, and bronchial tubes. The most suitable route may depend on the condition and disorder of the recipient. They may be administered alone or with suitable pharmaceutical carriers, and can be in solid or liquid form such as, tablets, capsules, powders, solutions, suspensions, or emulsions.

The active compounds of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 and 250 mg of active compound.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

As will be apparent to the skilled artisan, the peptide may be administered in the form of a pharmaceutical formulation comprising any of the above described peptides and a pharmaceutically acceptable carrier. Acceptable pharmaceutical carriers include solutions, suspensions, emulsions, excipients, powders, or stabilizers. The carrier should be suitable for the desired mode of delivery.

In addition, the pharmaceutical formulations may further comprise one or more pharmaceutically acceptable diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Examples of suspending agents include ethoxylated isostearyl alcohols, polyoxyethylene sorbitol and sorbitan esters, microcrystalline cellulose, aluminum metahydroxide, bentonite, agar—agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

In particularly preferred embodiments in which the peptides are administered in vivo, administration is carried out orally, topically, parenterally, intravenously, by intranasal instillation, by application to mucous membranes, rectally, or by combinations thereof.

In the case of ex vivo administration, the population of CD8+ T cells can be isolated from the subject and then contacted with the peptide ex vivo as described above to produce a population of CD8+ T cells with stimulated suppressive activity. The population of CD8+ T cells with stimulated suppressive activity is then introduced into the subject. In a typical embodiment, CD8+ T cells are isolated from a sample (e.g., biopsy, blood, plasma) taken from the subject. The cells are then expanded in culture, using, for example, cytokines, to enrich CD8+ T cells in the population. The enriched population is then contacted (usually multiple times) with the peptide of the present invention until an improvement in the suppressive activity of the CD8+ T cells is observed. The CD8+ T cell population with improved activity is then reintroduced into the subject, usually intravenously or by injection into the intestinal mucosa. As will be apparent to the skilled artisan, each of these steps can be carried out using standard techniques that are well known in the art. Those skilled in the art will also appreciate that other ex vivo techniques for contacting a CD8+ T cell population with an agent may also be used.

The peptides of the present invention may be administered alone or in combination with one or more other agents for treating Crohn's disease.

Yet another aspect of the present invention relates to a method of identifying candidate peptides suitable for treating Crohn's disease in a subject. This method involves providing a collection of candidate peptides from a CEACAM5 N-terminal domain. A population of CD8+ T cells having reduced suppressive activity is also provided. A peptide from the collection of peptides is contacted with the population of CD8+ T cells under conditions effective to permit interaction between them. Peptides which interact with the population, as a result of the contacting, are identified as candidate peptides potentially suitable for treating Crohn's disease in a subject. Their suitability can optionally be further evaluated in pre-clinical and/or clinical trials.

In at least one embodiment, the collection of peptides is an overlapping peptide library, for example, with an offset number of 5. The library can include multiple pools of peptides.

Preferably, the candidate peptides according to this aspect of the present invention have a minimum length of 4, 5, 6, 7, 8, 9, 10, 11, 12, 13, 14, or 15 amino acids and a maximum length of 4, 5, 6, 7, 8, 9, 10, 11, 12, 13, 14, or 15 amino acids. Combinations of each of these minimum and maximum lengths are expressly contemplated. In a preferred embodiment, the candidate peptides have 4-15 amino acids. In a preferred embodiment the candidate peptides have 4-10 amino acids. In a preferred embodiment the candidate peptides have 4-8 amino acids.

Suitable CEACAM5 N-terminal domains and CD8+ T cell populations according to this aspect of the present invention include those noted above.

As will be apparent to the skilled artisan, there are a number of suitable techniques for identifying peptides that interact with CD8+ T cells. These techniques including binding assays (e.g., like that described in Roda et al., Mucosal Immunol. 7(3): 615-24 (2014), which is hereby incorporated by reference in its entirety), LcK phosphorylation assays (e.g., like those described in Examples 3 and 5, infra), CD4+ cell proliferation assays (e.g., like those described in Examples 1, 4, and 6, infra), and assays that monitor one or more markers for suppressor T cells (see, e.g., Rabinowitz et al., Gastroenterology 144:601-12 (2013), which is hereby incorporated by reference in its entirety), including IL10 (e.g., as described in Example 7, infra).

Yet another aspect of the invention relates to a pharmaceutical formulation comprising a peptide from a CEACAM5 N-terminal domain and a pharmaceutically acceptable carrier, where the N-terminal domain consists essentially or consists of: (i) residues 45-70 of the amino acid sequence of SEQ ID NO: 1, (ii) residues 62-70 of the amino acid sequence of SEQ ID NO: 1, or (iii) residues 45-81 of the amino acid sequence of SEQ ID NO: 1.

Suitable peptides according to this aspect of the present invention include those noted above. In at least one embodiment, the pharmaceutical formulation does not comprise full-length CEACAM5. In at least one embodiment, the pharmaceutical formulation does not comprise a peptide having the amino acid sequence of SEQ ID NO: 1. In at least one embodiment, the pharmaceutical formulation does not comprise a peptide consisting of residues 70-81 of the amino acid sequence of SEQ ID NO: 1.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “pharmaceutically acceptable” means it is, within the scope of sound medical judgment, suitable for use in contact with the cells of humans and lower animals without undue toxicity, irritation, allergic response, and the like, and is commensurate with a reasonable benefit/risk ratio.

The pharmaceutical composition according to the present invention can be formulated for administration as described above. As will be apparent to the skilled artisan, the pharmaceutical formulation may be in the form of a combination therapy and include one or more other therapeutic agents in addition to the CEACAM5 N-terminal peptide. Preferably the one or more other therapeutic agents is another CEACAM5 N-terminal domain peptide and/or other therapeutic agent for Crohn's disease.

The present invention may be further illustrated by reference to the following examples.

### Examples

### Example 1—Stimulation of the Suppressive Activity of Lamina Propria CD8+ T Cells by Full-Length CEACAM5 (in Vitro Suppression Assay)

Crohn's disease lamina propria CD8+ T cells have previously been shown to have impaired suppressive function. The effect of CEACAM5 on the suppressive activity of CD8+ T cells was compared in Crohn's disease and non-Crohn's disease lamina propria cells.

Lamina propria lymphocytes were isolated from patients undergoing surgery for Crohn's disease (CD) or colon cancer (representing a normal (NL) T cell population). Lamina propria CD8+ T cells were isolated after overnight incubation and incubated with CEACAM5 peptide for 3 days. CD8+ T cells were then washed and co-cultured with CFSE-CD3/CD28-stimulated CD4+ T cells for 3 days. Suppression of CD4+ T cell proliferation was measured on the 7^{th} day.

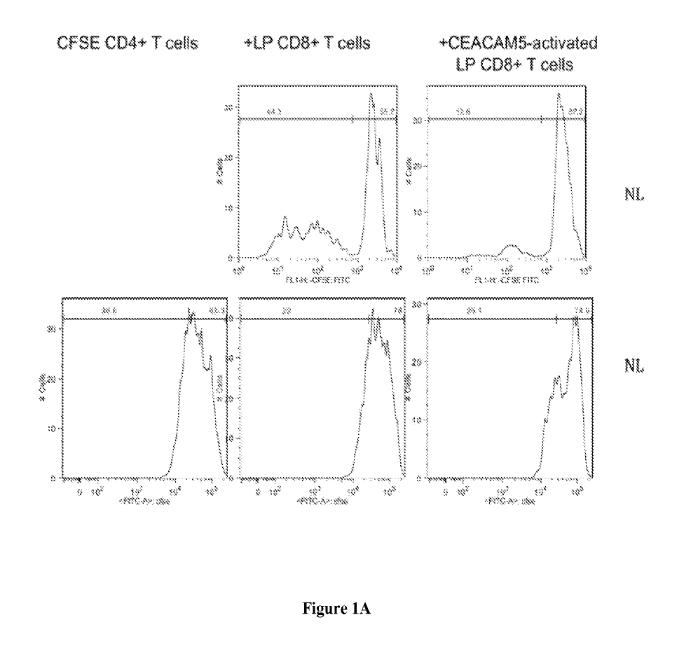

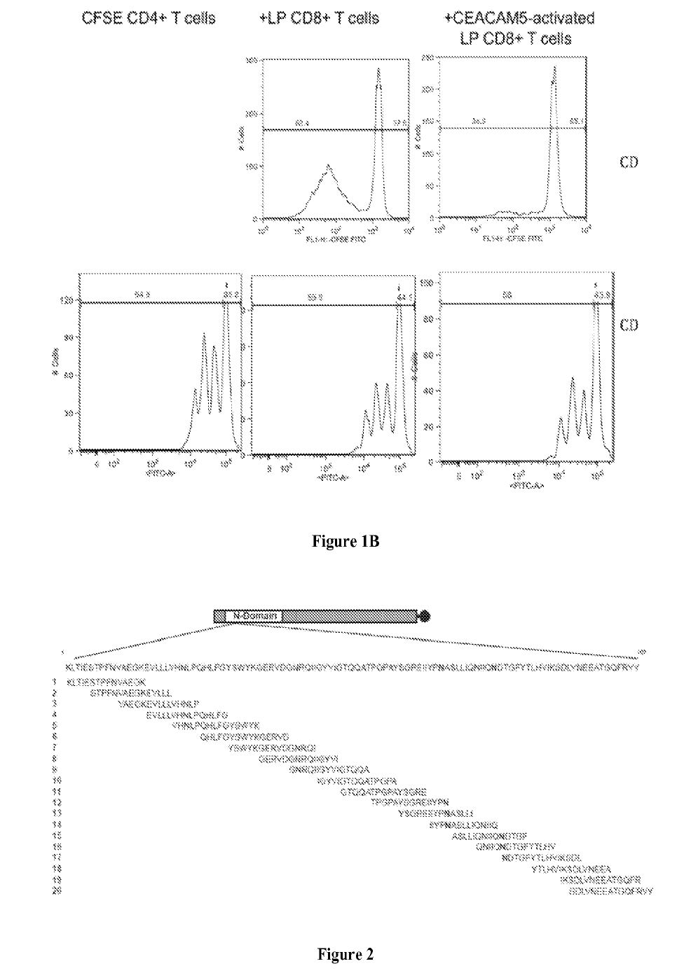

As shown in FIGS. 1A-B, CEACAM5-activated lamina propria CD8+ T cells have an increased suppressive function in the normal condition than in the absence of CEACAM5, and CEACAM5 stimulates the suppressive function of lamina propria CD8+ T cells in Crohn's disease.

### Example 2—CEACAM5 N-Domain Overlapping Peptide Library

A novel unique property of the CEACAM5 N-terminal domain, that of immunomodulation, was previously identified (Roda et al., Mucosal Immunol. 7(3): 615-24 (2014), which is hereby incorporated by reference in its entirety). To dissect the functional region of CEACAM5, an overlapping peptide library of its N-domain was generated. An overlapping peptide library is mostly used for linear and continuous epitope mapping. The aim is to generate a library of overlapping peptide sequences of specific length and specific offset that covers the N-domain of CEACAM5. As shown in FIG. 2, the library was generated by breaking the N-domain (residues 1-107 (SEQ ID NO: 1)) into many equal-length overlapping fragments with the offset number of 5 to limit the number of peptides, which resulted in an overlapping peptide library of 20 peptides (Table 1, infra). This library was further split into three pools: Pool 1 (peptides 1-6, Pool 2 (peptides 7-11), and Pool 3 (peptides 12-20).

TABLE 1
Overlapping Peptide Library.

| Peptide | SEQUENCE | SEQ ID NO |
|---|---|---|
| 1 | H-KLTIESTPFNVAEGK-OH | 2 |
| 2 | H-STPFNVAEGKEVLLL-OH | 3 |
| 3 | H-VAEGKEVLLLVHNLP-OH | 4 |
| 4 | H-EVLLLVHNLPQHLFG-OH | 5 |
| 5 | H-VHNLPQHLFGYSWYK-OH | 6 |
| 6 | H-QHLFGYSWYKGERVD-OH | 7 |
| 7 | H-YSWYKGERVDGNRQI-OH | 8 |
| 8 | H-GERVDGNRQIIGYVI-OH | 9 |
| 9 | H-GNRQIIGYVIGTQQA-OH | 10 |
| 10 | H-IGYVIGTQQATPGPA-OH | 11 |
| 11 | H-GTQQATPGPAYSGRE-OH | 12 |
| 12 | H-TPGPAYSGREIIYPN-OH | 13 |
| 13 | H-YSGREIIYPNASLLI-OH | 14 |
| 14 | H-IIYPNASLLIQNIIQ-OH | 15 |
| 15 | H-ASLLIQNIIQNDTGF-OH | 16 |
| 16 | H-QNIIQNDTGFYTLHV-OH | 17 |
| 17 | H-NDTGFYTLHVIKSDL-OH | 18 |
| 18 | H-YTLHVIKSDLVNEEA-OH | 19 |
| 19 | H-IKSDLVNEEATGQFR-OH | 20 |
| 20 | H-SDLVNEEATGQFRVY-OH | 21 |

Previous results had indicated that the N-domain of CEACAM5 is involved in the interaction with CD8α and in activation of peripheral blood CD8+ T cells. The residues between amino acid 70-81 of the N-domain have been determined to be crucial in this process. For all the experiments described in Examples 3-5 herein, the entire library of peptides, 3 pools of peptides, and/or each single peptide, have been used. Roda et al., Mucosal Immunol. 7(3): 615-24 (2014), which is hereby incorporated by reference in its entirety, showed that the glycosylation site within residue 70 and 81 is crucial. Consequently, Pool 1 included peptides covering a region from residues 1-33, Pool 2 from residues 30-65, and Pool 3 from residues 66-107 (which includes residues 70 and 81). The activity of four additional peptides, designated a, b, c, and d (Table 2, infra), was also evaluated to further characterize the amino acids involved in the functionality of CEACAM5.

TABLE 2
Peptides a-d.

| Peptide | SEQUENCE | SEQ ID NO |
|---|---|---|
| a | ERVDGNRQIIGYVIGTQQAT | 22 |
| b | NRQIIGYVIGTQQATPGPAY | 23 |
| c | GYVIGTQQATPGPAYSGREI | 24 |
| d | TQQATPGPAYSGREIITPNA | 25 |

### Example 3—Peripheral Blood CD8+ T Cell Activation by CEACAM5 N-Terminal Peptides (In Cell Western Blot LcK Phosphorylation Assay)

The library of peptides 1-20 was first evaluated by western blot for their ability to activate CD8+ T cells, as indicated by the ability to stimulate LcK phosphorylation (“pLcK”). Peripheral blood isolated CD8′ T cells (2×10⁶) were seeded in a 96-well flat-bottom plate and starved in RPMI serum free medium overnight at 37° C. The next day and prior to performing the experiments, the plate was placed on ice for 15 minutes. Cells were stimulated with OKT8 (5 μg/mL) (a known CD8+ T cell activator), purified CEACAM5 peptide (10 μg/mL), the entire library of peptides (10 μg/mL), Pool 1 (10 μg/mL), Pool 2 (10 μg/mL), Pool 3 (10 μg/mL), or each single N-domain peptide (10 μg/mL). Unstimulated cells were also evaluated for each condition. Prior to these experiments a titration curve was performed for the entire library of peptides. A range of concentration between 1 and 100 μg/mL was tested.

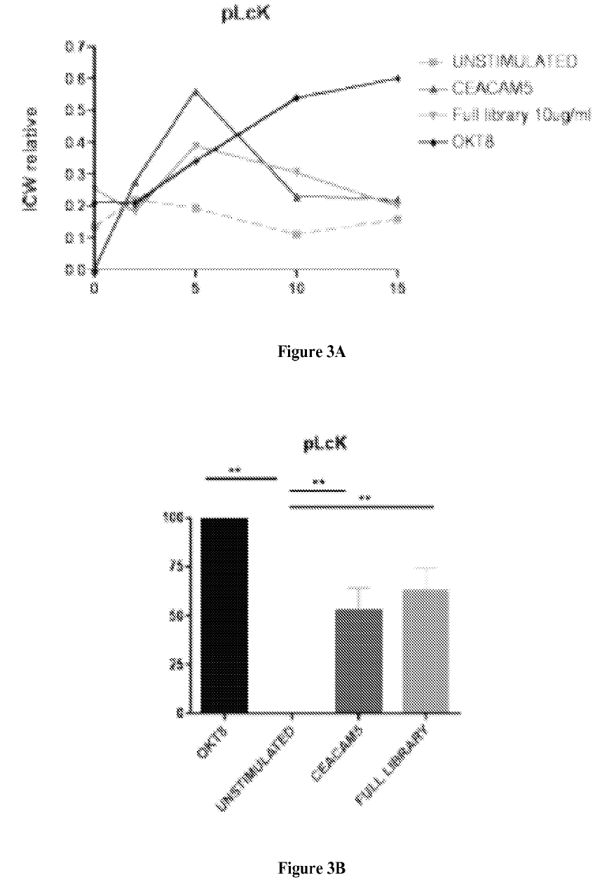

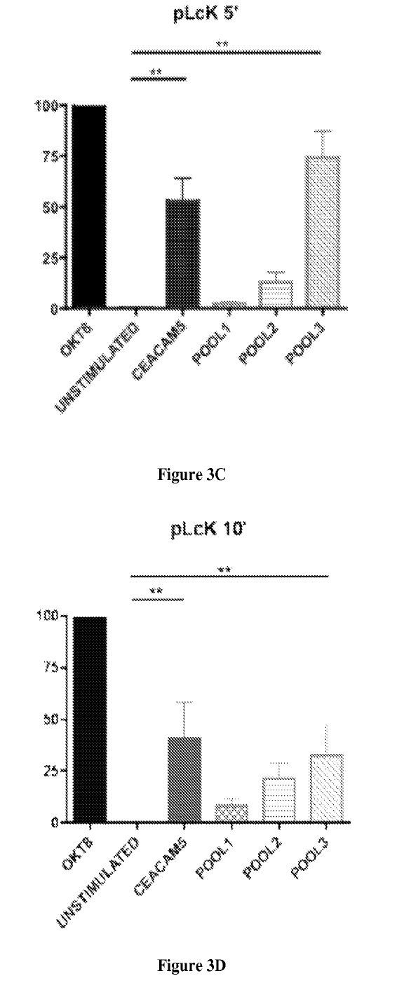

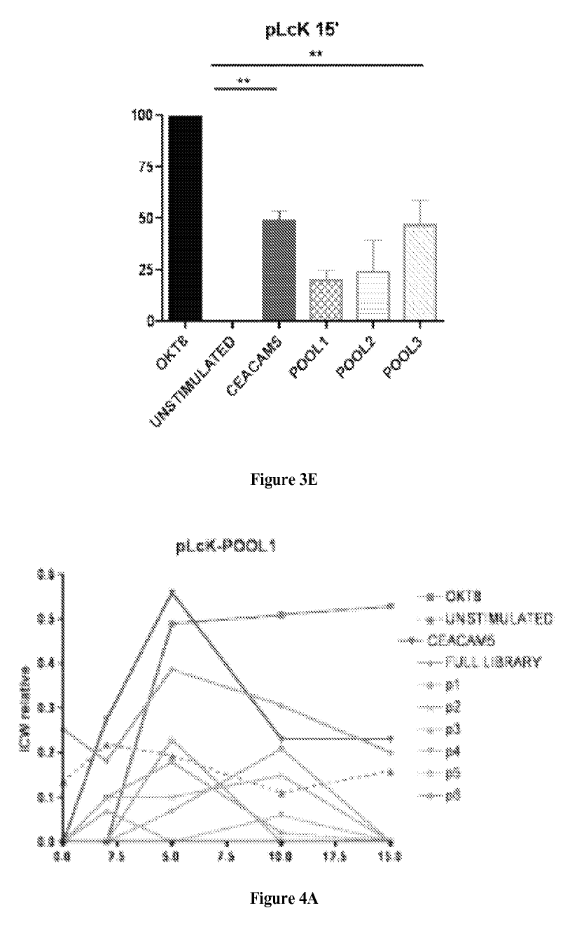

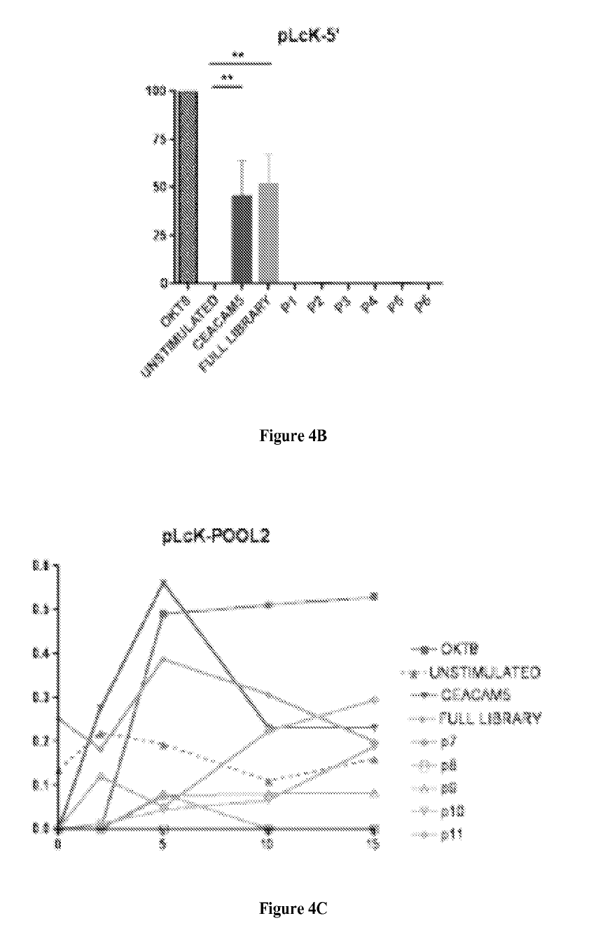

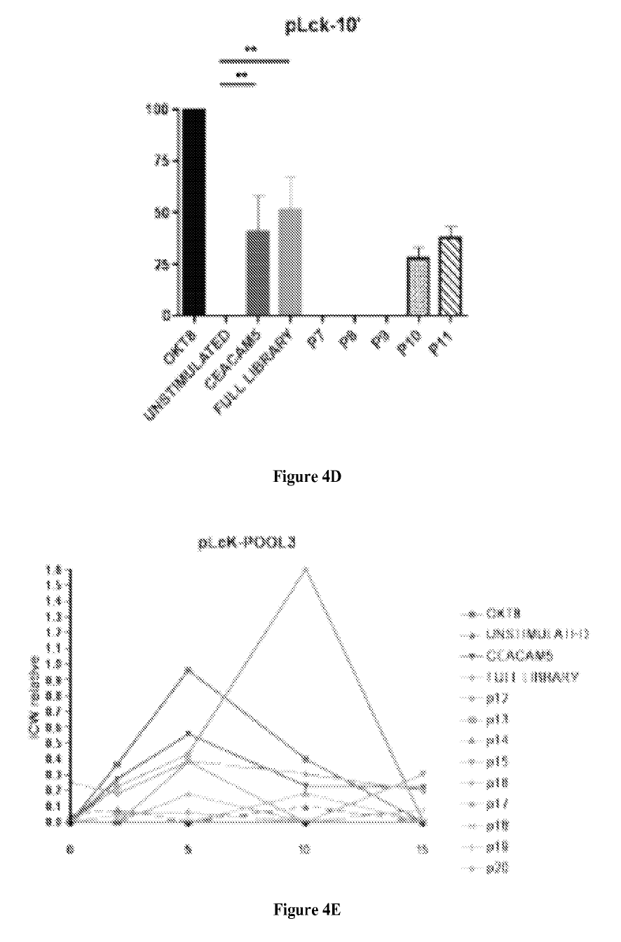

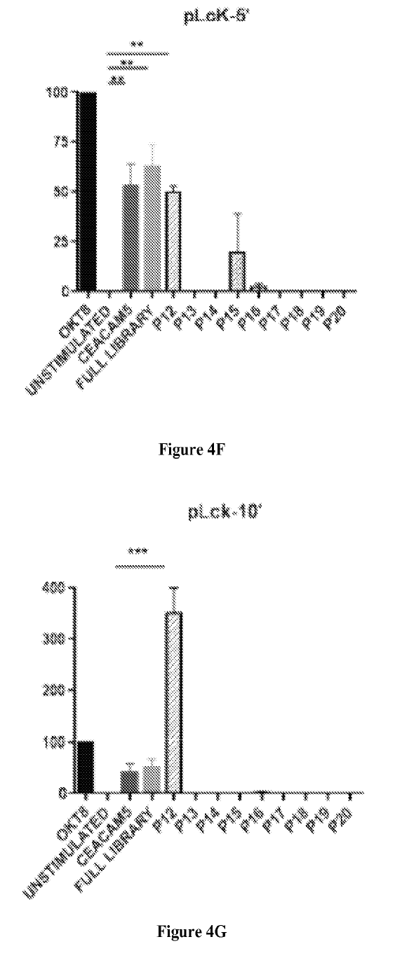

Stimulation was stopped using a 4% formaldehyde buffer for 20 minutes followed by centrifugation for 10 minutes at 1,500 rpm. Permeabilization was performed by washing the cells with 1×PBS containing 0.1% Triton X-100 for 5 minutes per wash. After permeabilization, cells were incubated for 1 hour with a blocking buffer (Odyssey Blocking buffer-LICOR) followed by the primary antibodies (goat anti-actin and rabbit anti-pLcK) overnight at 4° C. The next day, cells were washed 5 times with 1×PBS containing 0.1% Tween 20, incubated for 1 hour at room temperature with the secondary antibodies (IRDye 800CW donkey anti goat IgG, IRDye 680 CW donkey anti rabbit IgG), and then washed five times. Wells were dried and the plate was scanned using an Odyssey LiCOR system. The software ImageJ was used for quantification of pLcK. Each value was calculated using actin expression as a house keeping gene and taking into consideration any background noise. See FIGS. 3A-B (full-length CEACAM5 and the full library), FIGS. 3C-E (full-length CEACAM5 and pools 1-3), FIGS. 4A-B (full-length CEACAM5, the full library, and individual peptides 1-6), FIGS. 4C-D (full-length CEACAM5, the full library, and individual peptides 7-11), and FIGS. 4E-G (full-length CEACAM5, the full library, and individual peptides 12-20).

As shown in FIGS. 3A-E, Pool 3 is the best activator of CD8+ T cells among the 3 pools tested. FIG. 3A also demonstrates that the full N-terminal peptide library has the same efficacy as full length CEACAM5.

As shown in FIGS. 4A-G, peptides from Pool 1 had no effect on CD8+ T cell activation (FIGS. 4A-B), while peptides 10 and 11 from Pool 2 induced phosphorylation of LcK at after 10 minutes (FIGS. 4C-D) and peptide 12 from Pool 3 resulted in the greatest effect in the activation of CD8+ T cells (FIGS. 4E-G).

### Example 4—Stimulation of the Suppressive Activity of Peripheral Blood CD8+ T Cells by CEACAM5 N-Terminal Peptides (in Vitro Suppression Assay)

The full library of peptides (peptides 1-20) and Pools 1-3 were next evaluated for their ability to trigger CD8+ T cell suppressor activation, as indicated by the effect of stimulated peripheral blood CD8+ T cells on CFSE-CD3/CD28 stimulated CD4+ T cells proliferation. To identify the N-domain peptides that trigger the activation of CD8+ T cells with the suppressor phenotype, peripheral blood CD8⁺ T cells and peripheral blood CD4⁺ T cells were selected using CD8⁺ and CD4⁺ T-cell selection kits (Stemcell Technologies, Vancouver, Canada), respectively.

Peripheral blood CD8′ T cells (3×10⁶) were incubated for 3 days in the presence or absence of purified CEACAM5 peptide (10 μg/mL), the entire library of peptides (peptides 1-20) (10 μg/mL), Pool 1 (10 μg/mL), Pool 2 (10 μg/mL), or Pool 3 (10 μg/mL), added every other day for 3 days. Anti-CD3/CD28 beads were used as positive controls. Cells were then washed, counted, and added at a ratio of 1:1 (2×10⁶) to peripheral CD4⁺ T cells labeled with carboxyfluorescein succinimidyl ester (CFSE) (Invitrogen, CA), and either unstimulated or stimulated with anti-CD3/CD28 beads.

Proliferation of CD4⁺ T-cells measured by CFSE dilution was evaluated by flow cytometry. CD4+ T cells were labeled with PE anti-CD4 antibody. Percent suppression was calculated based on equation 1:

% Supp=100*[1−(%_{Exp}/%_{Control})] (1)

where:
%_{Supp}=percent suppression
%_{Exp}=the percentage of proliferating CD4+ T cells in the experimental population
% Control=the percentage of proliferating CD4+ T cells in the control population

As shown in FIGS. 5A-F, Pool 3 appeared to have the greatest suppression activity among the three pools tested.

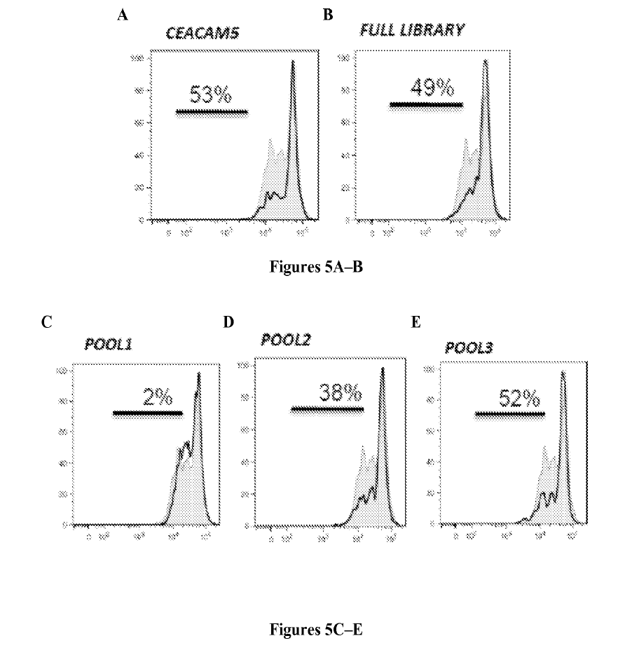

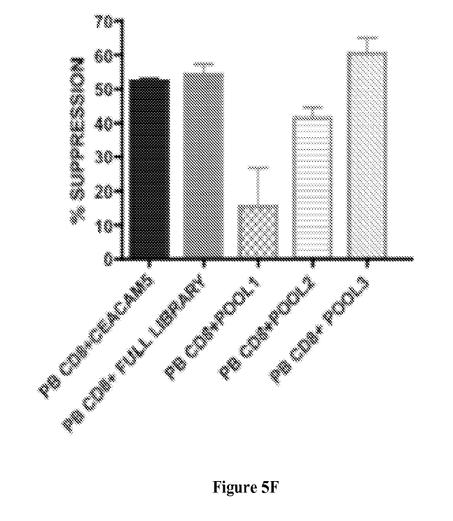

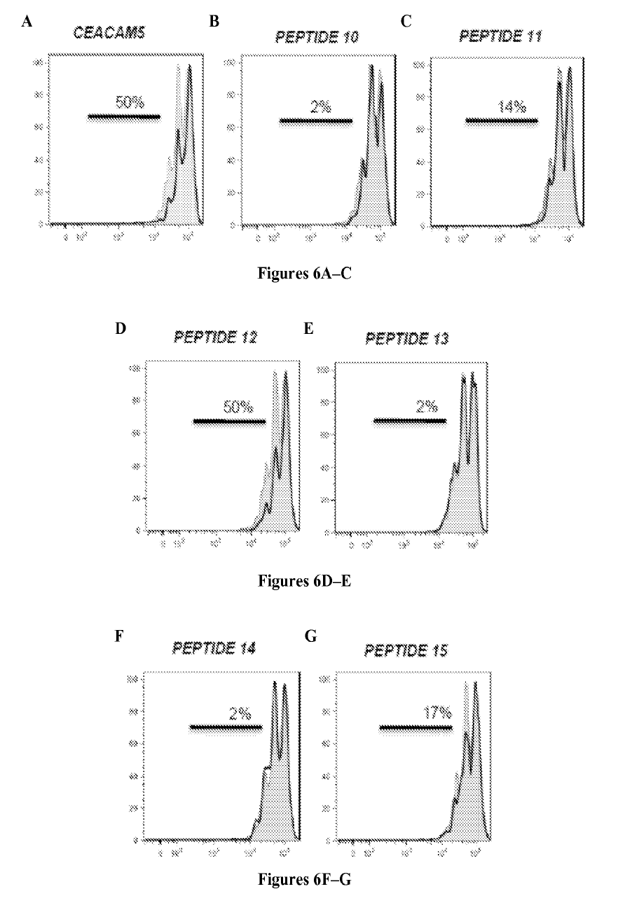

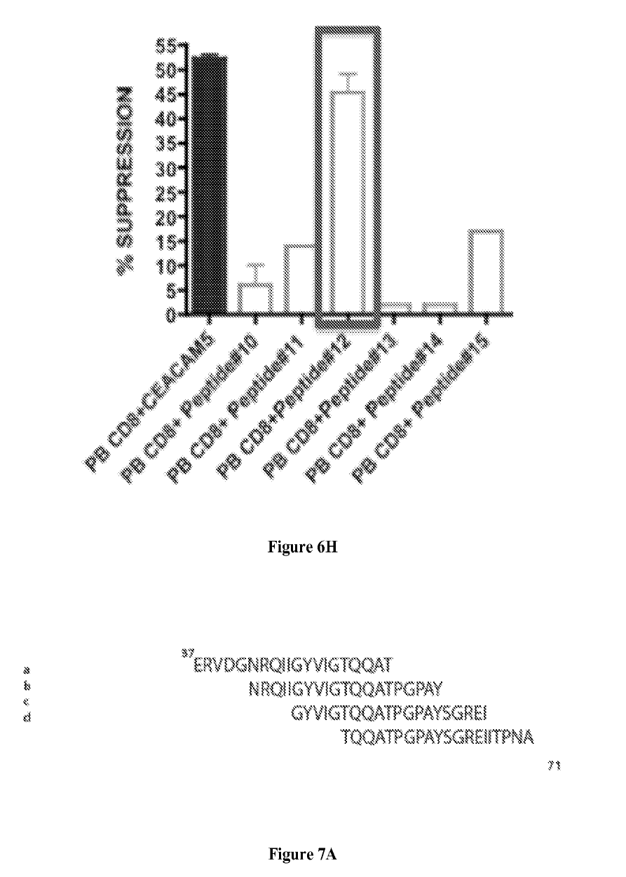

Next, peptides 10-15 were next evaluated using the same procedure, incubating the cells with each single peptide (10 μg/mL). These peptides were selected due to their activity in the phosphorylation assay. As shown in FIGS. 6A-H, peptide 12-activated CD8+ T cells acquired suppressor activity at the same extant as those activated with full-length CEACAM5. Peptides 13 and 15 were also shown to activate suppression, although to a more modest extent (˜10% suppressive activity).

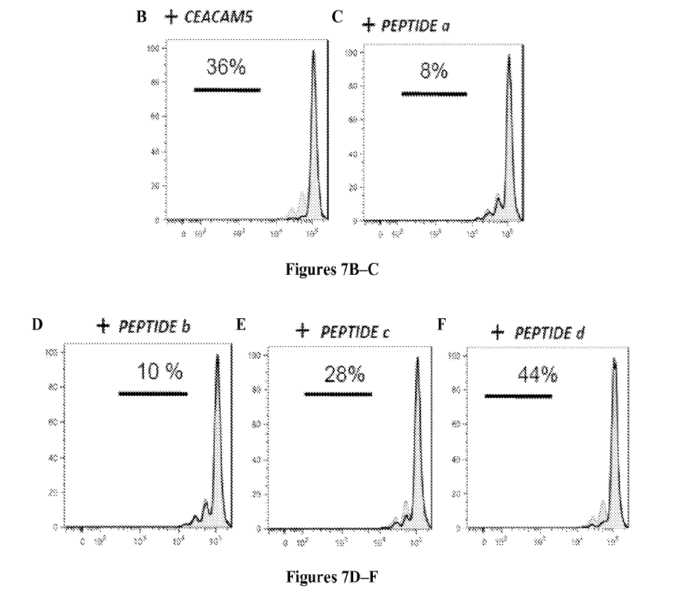

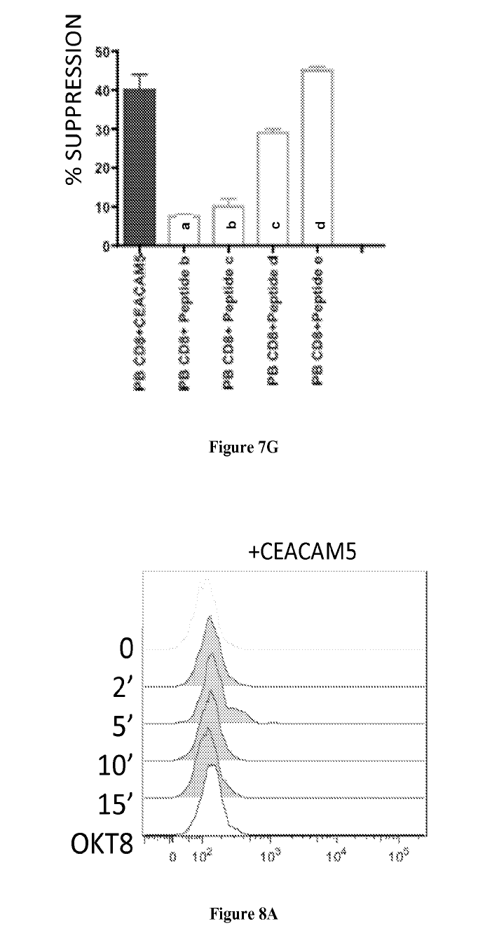

Finally, peptides a-d (see FIG. 7A) were also evaluated using the same procedure, incubating the cells with each single peptide (10 μg/mL). As shown in FIGS. 7B-G, peptide c-d-activated CD8+ T cells all acquired suppressor activity, with peptide d-activated cells acquiring suppressor activity to the same extent as cells activated with peptide 12. Notably, peptides c and d share sequences that are also present in peptide 12, which indicates that these sequences may be particularly efficacious in inducing the acquisition of the suppressor phenotype by CD8+ T cells.

### Example 5—Peripheral Blood CD8+ T Cell Activation by CEACAM5 N-Terminal Peptide 12 (Flow Cytometry LcK Phosphorylation Assay)

To confirm the ability of peptide 12 to activate CD8+ T cells, a second LcK phosphorylation assay was conducted using flow cytometry. CD8+ T cells were treated for different time points (2, 8, 10, 15 minutes) at 37° C. with H₂O₂ at 10 mM, CEACAM5 supernatant from PIPLC-293T cells expressing CEACAM5, and peptide 12 (20 μg/ml). Stimulation was stopped by fixing with 4% formaldehyde for 10 minutes at room temperature. Subsequently, cells were permeabilized with ice-cold methanol for 20 minutes, washed with PBS containing 0.2% FBS, and then stained with anti-phospho-Lck Ab (BD Biosciences, San Jose, Calif.). The cells were then re-suspended in PBS plus 0.2% FBS, data were acquired using the FACScan system (LSRFortessa, BD Biosciences), and analyzed with FlowJo analysis software (Ashland, Oreg.).

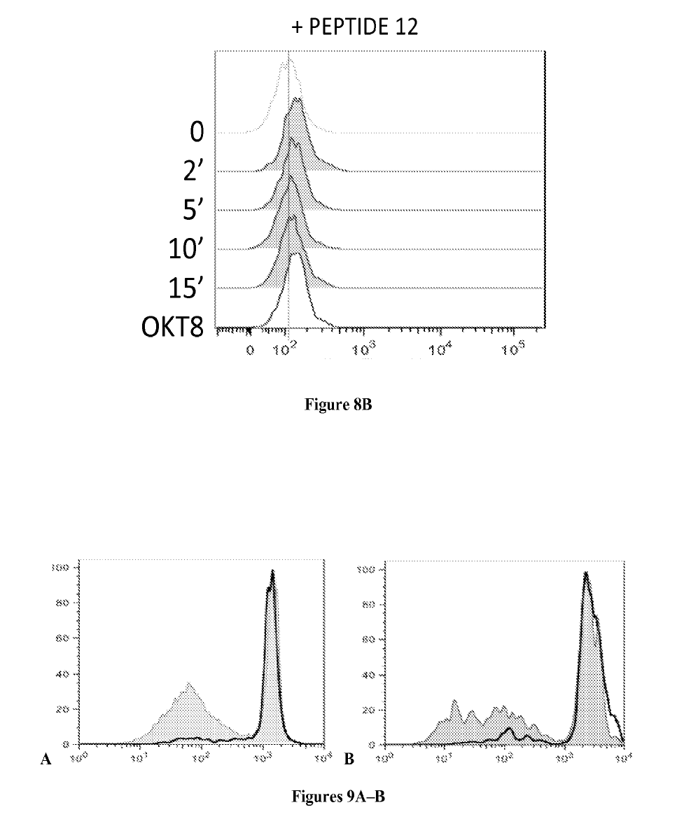

As shown in FIGS. 8A-B, the flow cytometry assay confirmed that CEACAM5 and peptide 12 induce phosphorylation of CD8-associated LcK kinase.

### Example 6—Stimulation of the Suppressive Activity of Lamina Propria CD8+ T Cells from Normal and Crohn's Disease Surgical Specimens by Full-Length CEACAM5 and Peptide 12 (in Vitro Suppression Assay)

The effect of CEACAM5 and peptide 12 on the suppressive activity of CD8+ T cells was compared in Crohn's disease and non-Crohn's disease lamina propria cells.

Lamina propria lymphocytes were isolated from patients undergoing surgery for Crohn's disease (CD) or colon cancer (representing a normal (NL) T cell population). Lamina propria CD8+ T cells were isolated after overnight incubation and incubated with CEACAM5 peptide or peptide 12 for 3 days. CD8+ T cells were then washed and co-cultured with CFSE-CD3/CD28-stimulated CD4+ T cells for 3 days. Suppression of CD4+ T cell proliferation was measured on the 7^{th} day.

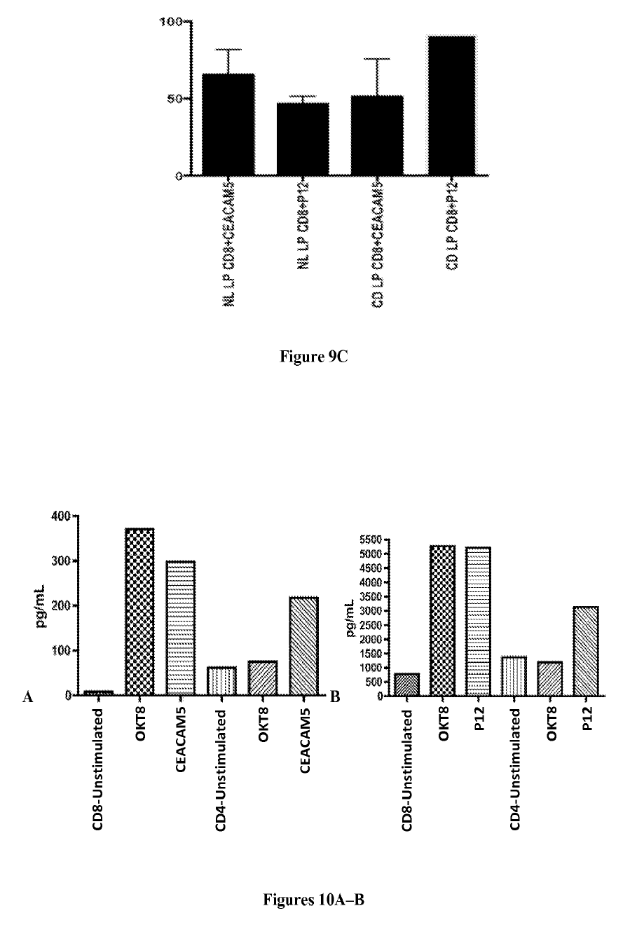

As shown in FIGS. 9A-C, Crohn's disease lamina propria CD8+ cells acquire a suppressor phenotype in presence of both CEACAM5 and peptide 12 (63% and 64%, respectively).

### Example 7—Cytokine Production in Lamina Propria CD8+ T Cells

Cytokine production in lamina propria CD8+ T cells was evaluated. Normal lamina propria CD8+ and CD4+(control) T cells were cultured for 3 days in presence of OKT8 (control) or either CEACAM5 or peptide 12. The supernatant was then assessed for cytokine production. It was observed that lamina propria CD8+ T cells produce increased IL10 in presence of CEACAM5 or peptide 12.

### Discussion of Examples 1-7

As described in Example 1, CEACAM5 is able to stimulate the suppressive activity of CD8+ T cells in Crohn's disease. As shown in Examples 2-5, the CEACAM5 N-domain was dissected to identify which amino acid residues are responsible for its immunosuppressive function. To this purpose, a CEACAM5 N domain overlapping peptide library of small peptides (˜10 amino acids with offset of 5) was generated and their immunoregulatory function was tested to select those able to stimulate the impaired suppressive activity in Crohn's disease. The ability of each single peptide and pool of peptides to: I) activate CD8+ T cells by inducing phosphorylation of CD8αassociated LcK kinase and to II) induce the suppressive phenotype in peripheral isolated CD8+ T cells was evaluated. Among the 24 peptides tested, several were able to activate and induce a suppressive phenotype on peripheral CD8+ T cells from healthy donor subjects. Two peptides containing residues where the glycosylation site is located appeared to activate CD8+ T cells at a greater extent than CEACAM5. Interestingly, some of the pools or peptides tested showed reduced activity, and they may have an inhibitory function. As shown in Example 6, the ability of peptide 12 to induce the suppressor phenotype was further confirmed by comparing its effect to that of CEACAM5 in lamina propria cells from normal and Crohn's disease patients. As shown in Example 7, production of cytokine IL10, a known suppressor cytokine, is increased in cells treated with CEACAM5 or peptide 12, demonstrating that IL10 can be used to monitor the effect of these peptides on CD8+ T cell suppressor activity.

These Examples demonstrate that small peptides derived from the N-terminus of the CEACAM5 protein stimulate the suppressive activity of lamina propria CD8+ T cells derived from Crohn's disease patients. While examining the activation of CD8+ T cells incubated with individual small peptides derived from the CEACAM5 N-terminal domain, the ability of CD8+ T cells to mediate suppression of CD4+ T cell proliferation was also investigated. The data presented demonstrate that peptides derived from the CEACAM5 N-terminus have immunomodulatory effects and are able to stimulate the suppressive activity of CD8+ T cells isolated from Crohn's disease patients. These findings indicate an important role for short CEACAM5 N-terminal peptides in the treatment of patients with Crohn's disease, where lack of suppressive CD8+ T cells as a consequence of a defect in CEACAM5 expression in intestinal epithelial cells has been described. Specifically, it is expected that CEACAM5 N-terminal peptides and activating peptide fragments thereof, whether administered as a pharmaceutical composition, expressed via gene expression vector, or otherwise synthesized, can be used to treat intestinal diseases or disorders relating to a defect in CEACAM5 expression, such as Crohn's disease, as well as manage symptoms thereof and heal intestinal tissue damaged by such disorders.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of stimulating suppressive activity in CD8+ T cells, said method comprising:
providing a population of CD8+ T cells having reduced suppressive activity and
contacting the provided population of CD8+ T cells with a peptide from a CEACAM5 N-terminal domain under conditions effective to stimulate suppressive activity in the provided population of CD8+ T cells.

2. The method of claim 1, wherein the CEACAM5 N-terminal domain consists essentially of: (i) residues 45-70 of the amino acid sequence of SEQ ID NO: 1, (ii) residues 70-81 of the amino acid sequence of SEQ ID NO: 1, (iii) residues 62-70 of the amino acid sequence of SEQ ID NO: 1, or (iv) residues 45-81 of the amino acid sequence of SEQ ID NO: 1.

3-6. (canceled)

7. The method of claim 1, wherein the peptide is selected from the group consisting of: (i) a peptide consisting essentially of four to about 26 contiguous amino acids of residues 45-70 of the amino acid sequence of SEQ ID NO: 1, (ii) a peptide consisting essentially of four to about 12 contiguous amino acids of residues 70-81 of the amino acid sequence of SEQ ID NO: 1, (iii) a peptide consisting essentially of four to about 9 contiguous amino acids of residues 62-70 of the amino acid sequence of SEQ ID NO: 1, and (iv) a peptide consisting essentially of four to about 37 contiguous amino acids of residues 45-81 of the amino acid sequence of SEQ ID NO: 1.

8-11. (canceled)

12. The method of claim 1, wherein the peptide has 4-15 amino acids.

13. The method of claim 1, wherein the provided population of CD8+ T cells is a population of regulatory T cells.

14. A method of treating Crohn's disease in a subject, said method comprising:
selecting a subject with Crohn's disease and
administering a peptide from a CEACAM5 N-terminal domain to a population of CD8+ T cells of the subject, wherein the population of CD8+ T cells has reduced suppressive activity, under conditions effective to treat Crohn's disease in the subject.

15. The method of claim 14, wherein the CEACAM5 N-terminal domain consists essentially of: (i) residues 45-70 of the amino acid sequence of SEQ ID NO: 1, (ii) residues 70-81 of the amino acid sequence of SEQ ID NO: 1, (iii) residues 62-70 of the amino acid sequence of SEQ ID NO: 1, or (iv) residues 45-81 of the amino acid sequence of SEQ ID NO: 1.

16-19. (canceled)

20. The method of claim 14, wherein the peptide is selected from the group consisting of: (i) a peptide consisting essentially of four to about 26 contiguous amino acids of residues 45-70 of the amino acid sequence of SEQ ID NO: 1, (ii) a peptide consisting essentially of four to about 12 contiguous amino acids of residues 70-81 of the amino acid sequence of SEQ ID NO: 1, (iii) a peptide consisting essentially of four to about 9 contiguous amino acids of residues 62-70 of the amino acid sequence of SEQ ID NO: 1, and (iv) a peptide consisting essentially of four to about 37 contiguous amino acids of residues 45-81 of the amino acid sequence of SEQ ID NO: 1.

21-24. (canceled)

25. The method of claim 14, wherein the peptide has 4-10 amino acids.

26. The method of claim 14, wherein said administering is carried out in vivo.

27. (canceled)

28. The method of claim 14, wherein said administering is carried out ex vivo.

29. The method of claim 28 further comprising:
isolating the population of CD8+ T cells with reduced suppressive activity from the subject,
contacting the population of CD8+ T cells with reduced suppressive activity with the peptide to produce a population of CD8+ T cells with stimulated suppressive activity, and
introducing into the subject the population of CD8+ T cells with stimulated suppressive activity.

30. The method of claim 29, wherein said introducing is carried out intravenously or by injection into the intestinal mucosa.

31. A method of identifying candidate peptides potentially suitable for treating Crohn's disease in a subject, said method comprising:
providing a collection of candidate peptides from a CEACAM5 N-terminal domain;
providing a population of CD8+ T cells having reduced suppressive activity;
contacting a peptide from the collection of peptides with the population of CD8+ T cells under conditions effective to permit interaction between them; and
identifying peptides which interact with the population, as a result of said contacting, as candidate peptides potentially suitable for treating Crohn's disease in a subject.

32-42. (canceled)

43. A pharmaceutical formulation comprising a peptide from a CEACAM5 N-terminal domain and a pharmaceutically acceptable carrier, wherein the N-terminal domain consists essentially of: (i) residues 45-70 of the amino acid sequence of SEQ ID NO: 1, (ii) residues 62-70 of the amino acid sequence of SEQ ID NO: 1, or (iii) residues 45-81 of the amino acid sequence of SEQ ID NO: 1.

44. (canceled)


SEQUENCE LISTING (26 sequences)

SEQ ID NO: 1 - protein, 107 aa, Artificial
  CEACAM5 N-terminal domain
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVY

SEQ ID NO: 2 - protein, 15 aa, Artificial
  Fragment of the CEACAM5 N-terminal domain
KLTIESTPFNVAEGK

SEQ ID NO: 3 - protein, 15 aa, Artificial
  Fragment of the CEACAM5 N-terminal domain
STPFNVAEGKEVLLL

SEQ ID NO: 4 - protein, 15 aa, Artificial
  Fragment of the CEACAM5 N-terminal domain
VAEGKEVLLLVHNLP

SEQ ID NO: 5 - protein, 15 aa, Artificial
  Fragment of the CEACAM5 N-terminal domain
EVLLLVHNLPQHLFG

SEQ ID NO: 6 - protein, 15 aa, Artificial
  Fragment of the CEACAM5 N-terminal domain
VHNLPQHLFGYSWYK

SEQ ID NO: 7 - protein, 15 aa, Artificial
  Fragment of the CEACAM5 N-terminal domain
QHLFGYSWYKGERVD

SEQ ID NO: 8 - protein, 15 aa, Artificial
  Fragment of the CEACAM5 N-terminal domain
YSWYKGERVDGNRQI

SEQ ID NO: 9 - protein, 15 aa, Artificial
  Fragment of the CEACAM5 N-terminal domain
GERVDGNRQIIGYVI

SEQ ID NO: 10 - protein, 15 aa, Artificial
  Fragment of the CEACAM5 N-terminal domain
GNRQIIGYVIGTQQA

SEQ ID NO: 11 - protein, 15 aa, Artificial
  Fragment of the CEACAM5 N-terminal domain
IGYVIGTQQATPGPA

SEQ ID NO: 12 - protein, 15 aa, Artificial
  Fragment of the CEACAM5 N-terminal domain
GTQQATPGPAYSGRE

SEQ ID NO: 13 - protein, 15 aa, Artificial
  Fragment of the CEACAM5 N-terminal domain
TPGPAYSGREIIYPN

SEQ ID NO: 14 - protein, 15 aa, Artificial
  Fragment of the CEACAM5 N-terminal domain
YSGREIIYPNASLLI

SEQ ID NO: 15 - protein, 15 aa, Artificial
  Fragment of the CEACAM5 N-terminal domain
IIYPNASLLIQNIIQ

SEQ ID NO: 16 - protein, 15 aa, Artificial
  Fragment of the CEACAM5 N-terminal domain
ASLLIQNIIQNDTGF

SEQ ID NO: 17 - protein, 15 aa, Artificial
  Fragment of the CEACAM5 N-terminal domain
QNIIQNDTGFYTLHV

SEQ ID NO: 18 - protein, 15 aa, Artificial
  Fragment of the CEACAM5 N-terminal domain
NDTGFYTLHVIKSDL

SEQ ID NO: 19 - protein, 15 aa, Artificial
  Fragment of the CEACAM5 N-terminal domain
YTLHVIKSDLVNEEA

SEQ ID NO: 20 - protein, 15 aa, Artificial
  Fragment of the CEACAM5 N-terminal domain
IKSDLVNEEATGQFR

SEQ ID NO: 21 - protein, 15 aa, Artificial
  Fragment of the CEACAM5 N-terminal domain
SDLVNEEATGQFRVY

SEQ ID NO: 22 - protein, 20 aa, Artificial
  Fragment of the CEACAM5 N-terminal domain
ERVDGNRQIIGYVIGTQQAT

SEQ ID NO: 23 - protein, 20 aa, Artificial
  Fragment of the CEACAM5 N-terminal domain
NRQIIGYVIGTQQATPGPAY

SEQ ID NO: 24 - protein, 20 aa, Artificial
  Fragment of the CEACAM5 N-terminal domain
GYVIGTQQATPGPAYSGREI

SEQ ID NO: 25 - protein, 20 aa, Artificial
  Fragment of the CEACAM5 N-terminal domain
TQQATPGPAYSGREIITPNA

SEQ ID NO: 26 - protein, 9 aa, Artificial
  Fragment of the CEACAM5 N-terminal domain
SGREIIYPN